(19) United States
(12) Patent Application Publication
(10) Pub. No.: US 2016/0286974 A1
(43) Pub. Date: Oct. 6, 2016

(54) SNORING RECOGNITION AND ELECTRIC POWER ADJUSTMENT

(71) Applicant: Dennis M. Boyd, St. Louis, MO (US)

(72) Inventor: Dennis M. Boyd, St. Louis, MO (US)

(21) Appl. No.: 14/675,713

(22) Filed: Mar. 31, 2015

(51) Int. Cl. A47C 27/08; A61B 5/00; A47C 20/08; A47C 27/10; A61F 5/56

(52) U.S. Cl. CPC A47C 27/083; A47C 27/10; A61F 5/56; A47C 20/08; A61B 5/6892

(57) ABSTRACT

A system and method provide for reducing snoring by a user sleeping on a mattress. The system comprising a triggering device configured to identify a snoring event, and an adjustable base portion for supporting the mattress, the adjustable base portion having at least one articulation plate configured to move the mattress to change the sleeping position of the user sleeping on the mattress, in response to the identification of the snoring event by the triggering device

### FIELD

The present disclosure relates to snoring recognition and electric power adjustment.

### BACKGROUND

This section provides background information related to the present disclosure which is not necessarily prior art.

Snoring may disturb persons sleeping and may cause sleep fragmentation. Snoring is also often a social problem by negatively affecting the sleeping conditions of a partner who shares the mattress with the snorer. There is a significant demand for a system and a method to reduce a user's snoring by changing his or her sleeping position in the event that snoring is detected.

### SUMMARY

This section provides a general summary of the disclosure, and is not a comprehensive disclosure of its full scope or all of its features.

Embodiments of the present disclosure provide a system and a method to detect a snoring event and reduce snoring of a user sleeping on a mattress by changing his or her sleeping position.

A preferred embodiment of a system for reducing snoring by a user sleeping on a mattress according to the principles of the present disclosure includes a triggering device configured to identify a snoring event and an adjustable base portion for supporting the mattress. The adjustable base portion has at least one articulation plate configured to move the mattress to change the sleeping position of the user sleeping on the mattress in response to the identification of the snoring event by the triggering device.

The triggering device preferably further includes a sensing pad on the mattress. The sensing pad is preferably disposed on the mattress.

According to another embodiment, the mattress preferably includes at least one air filled chamber, and the triggering device preferably includes at least one sensor in the at least one air filled chamber. The sensor preferably further includes a pressure sensor for measuring changes in the pressure in the at least one air chamber.

In an alternate embodiment, the triggering device preferably includes at least one microphone and a processor for processing signals picked up by the at least one microphone. The at least one microphone can preferably include a left microphone and a right microphone disposed on the left and right sides of the mattress. The processor is preferably configured to process the signals picked up by the left and right microphone to determine on which side of the mattress the snoring event is occurring, and to move only the side of the mattress on which the snoring event is occurring.

In still another embodiment, the adjustable base portion preferably has multiple articulation plates. For example, the multiple articulation plates can be a head articulation plate disposed adjacent a head section of the adjustable base portion, a left articulation plate disposed adjacent a left section of the adjustable base portion, and a right articulation plate disposed adjacent a right section of the adjustable base portion. These articulation plates are configured to move the head, left, and right portions of the mattress respectively. When detecting a snoring event by the triggering device, the adjustable base portion is preferably operated to raise one or more articulation plate to elevate one or more portions of the mattress and thereby change the sleeping position of the user sleeping on the mattress.

The method of detecting and reducing a user's snoring preferably further includes detecting another or a subsequent snoring event, further moving one or more articulation plate(s) to move the mattress and thereby change the sleeping position of the user.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic diagram of a system for reducing snoring by a user sleeping on a mattress in accordance with a preferred embodiment of the present disclosure.

FIG. 2 is a schematic diagram of the system having some example triggering devices of FIG. 1.

FIG. 3 is a schematic diagram of the system having articulation plates of FIG. 1.

FIGS. 4 and 5 are perspective views of the adjustable base having different number of articulation plates of FIG. 3.

FIG. 6 is a flow chart of one possible method implemented by a system for reducing snoring by a user sleeping on a mattress.

Corresponding reference numerals indicate corresponding parts throughout the several views of the drawings.

### DETAILED DESCRIPTION

Example embodiments will now be described more fully with reference to the accompanying drawings.

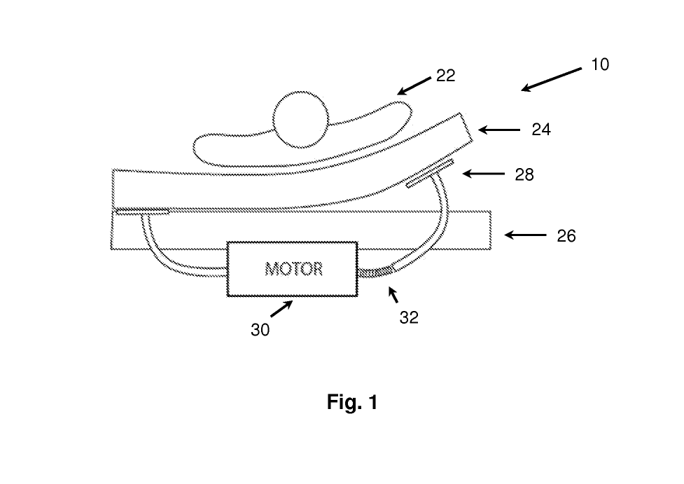

A preferred embodiment of a system 10 for reducing snoring by a user 22 sleeping on a mattress 24, in accordance with the principles of the present disclosure is indicated generally as 10 in FIG. 1.

The system 10 includes an adjustable base portion 26 for supporting the mattress 24. The adjustable base portion 26 further includes at least one articulation plate 28. The at least one articulation plate 28 can be moved by a motor 30 through a screw mechanism 32.

The system 10 preferably further includes a triggering device configured to identify a snoring event. In response to the identification of the snoring event, the articulation plate 28 is configured to move the mattress 24 to change the sleeping position of the user 22 sleeping on the mattress 24 for reducing snoring.

The snoring event can be identified by many different triggering devices. One preferred embodiment of a triggering device is for use with an air mattress. The mattress preferably includes at least one air filled chamber, and the triggering device preferably includes at least one sensor in the at least one air filled chamber. In this embodiment, the sensor may preferably further include a pressure sensor for measuring changes in the pressure in the at least one air chamber. The pressure sensor is preferably sensitive enough to distinguish different snoring patterns. The output from the pressure sensor can be processed to recognize a pattern of pressure change that corresponds to a snoring event, the pattern may be a single pressure change, or more likely a series of pressure changes, and thus the processing may account for the value of the pressure changes and their spacing

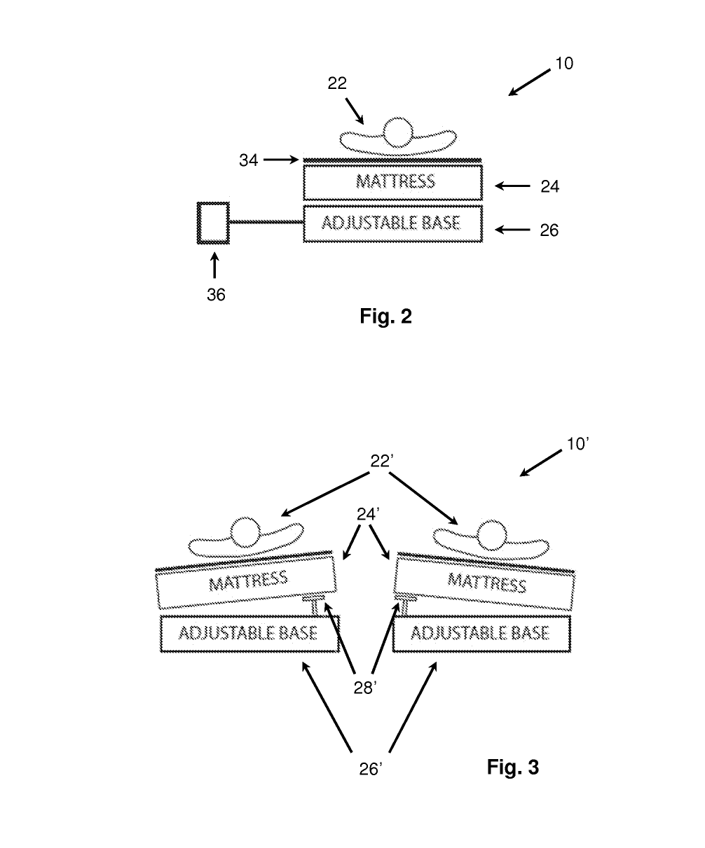

According to another embodiment, a triggering device of the system 10 shown in FIG. 2 includes a sensing pad 34. The sensing pad 34 is preferably disposed on top of the mattress 24 to sense stilted breathing or snoring.

Further, a triggering device most preferably includes at least one microphone 36 (shown in FIG. 2) and a processor for processing signals picked up by the microphone(s) 36. The output from the microphone(s) can be processed to recognize a pattern of sound that corresponds to a snoring event. The pattern may include the sound frequency, volume, duration, and spacing, some or all of which are used to recognize snoring. The processing can include comparing the sensed sound to factory set standards, or to personal standards acquired by the microphone over one or more nights. The processor is preferably programmed not only to identify a snoring event, but to identify the snorer when there are multiple occupants of the bed so that corrective action is taken on the correct side. The processor preferably can identify the snorer by processing the sound to determine relative distance from the microphone(s), or by the sound frequency or other differences in the sound patter, or by comparison with actual samples of snoring obtained for example during previous use of the bed.

An example of one way a processor can be programmed to identify a snoring event is as follows: The processor can be programmed to identify sound that is repeated intermittently a minimum of predetermined number (e.g., three) of times, with an pause between each sound of at least a predetermined minimum and or maximum period (e.g., between about 0.25 seconds and about 7.0 seconds), that is over a minimum threshold (e.g. 10 decibels), then the noise pattern would qualify as a “snore”. I have never seen a definition of a “snore” before but I think this should cover virtually all snore patterns. An additional qualifier may be added to restrict the definition to one in which the sounds would need to be measured in terms of duration (time) as well as decibels (volume of sound). Snoring patterns tend to be relatively consistent in decibels, length of sound emitted, and pauses between sounds. For example the variance could be determined and expressed as a percentage of the previous noise. If the percentage was exceeded on one of the three snores (or pauses), it would not qualify as a snore. Snoring is generally caused on the inhale portion of the breathing cycle.

A typical cycle may be as follows:
Inhale—snore is audible—30 decibels—duration 2.5 seconds
Exhale—respiration is not significantly audible—5 decibels—3 seconds
Inhale—snore is audible—29 decibels—duration 2.4 seconds
Exhale—respiration is not significantly audible—4 decibels—2.9 seconds
Inhale—snore is audible—31 decibels—duration 2.6 seconds
Exhale—respiration is not significantly audible—4 decibels—2.8 seconds
Inhale—snore is audible—30 decibels—duration 2.5 seconds

By setting the sound threshold, sound duration, and spacing, the processor can distinguish between snoring events and other sound events. Rather than minimums and maximums, varies parameters can be set, and sounds disqualified if they vary from the set parameters by more than a predetermined threshold, for example 25%, or the software can implement both a threshold requirement, and a variance requirement.

The thresholds and variances can be established and preprogrammed at the factory, and used permanently or used simply as defaults. The software can process user snoring sounds and set the thresholds and variances based upon actual user data. The processor can also store recurring back ground noise and either by comparison or filtering, exclude such noises to reduce the number of false identifications of snoring events.

The microphone(s) 36 may preferably include a left microphone and a right microphone disposed on each side of the mattress. The processor is preferably configured to process the signals picked up by the left and right microphones to determine on which side of the mattress the snoring event is occurring so that the system 10 may be operated to move only the side of the mattress 24 on which the snoring event is occurring. For example, when there are two users sleeping on the mattress 24, snoring patterns from each user can be recorded so that when the snoring was detected the system 10 would know which user initiated the snoring by comparing to the recorded snoring patterns so that the correct side of the mattress could be articulated. Statistics have shown that 85% of all adjustable mattresses sold are used as two twin extra long beds placed side by side to form a king size bed. Each side is operated independently of the other. The snoring detection needs to isolate the user doing the snoring so that it knows which side of the mattress to focus on in order to correct the sleep posture of that particular individual.

The different types of triggering devices provided herein can be used alone or in combination.

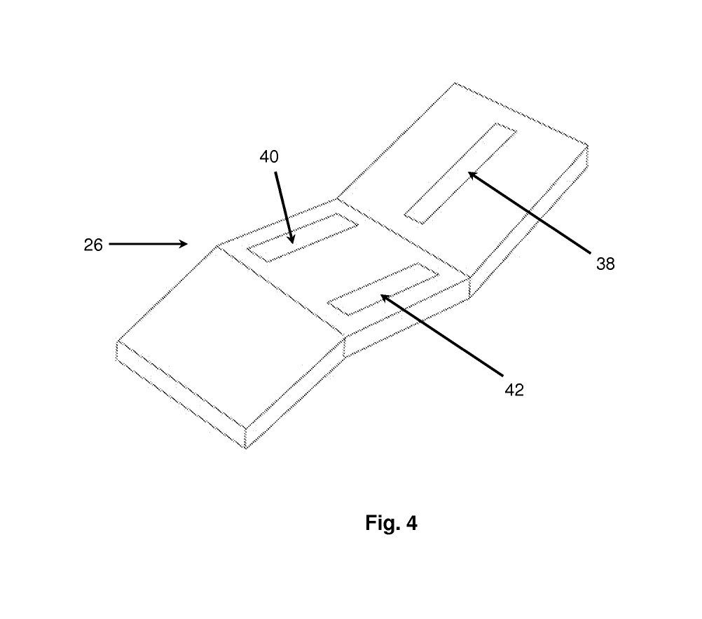

When snoring is properly detected and identified with the correct side of the mattress 24′, the articulation plate(s) 28′ of the correct side of the mattress 24′ may be preferably raised to achieve a comfortable sleeping angle as shown in FIG. 3.

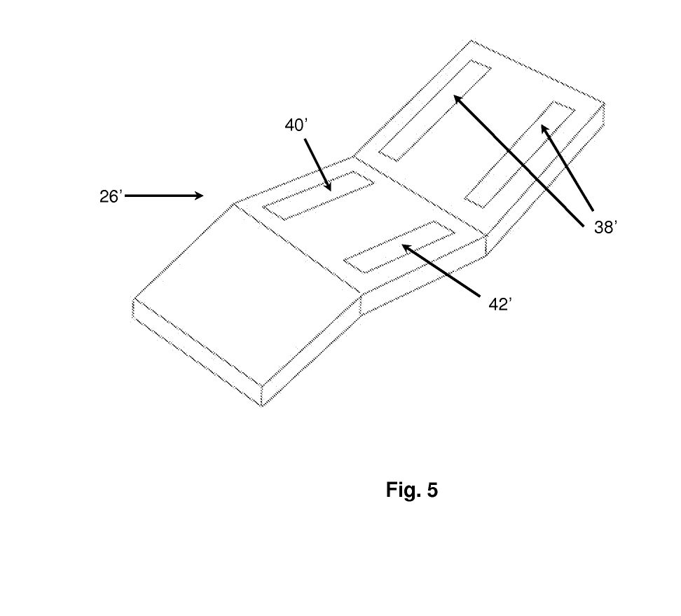

The adjustable base portion 26 preferably includes multiple articulation plates as shown in FIG. 4. For example, the multiple articulation plates can be at least one head articulation plate 38 disposed adjacent a head section of the adjustable base portion, a left articulation plate 40 disposed adjacent a left section of the adjustable base portion 26, and a right articulation plate 42 disposed adjacent a right section of the adjustable base portion 26. These articulation plates are configured to move head, left, and/or right portions of the mattress respectively. When detecting a snoring event by the triggering device, the adjustable base portion 26 can be preferably operated to raise one or more articulation plate to elevate one or more portions of the mattress and thereby change the sleeping position of the user sleeping on the mattress for the purpose of reducing snoring.

In still another embodiment, the at least one head articulation plate 38′ may include a left head articulation plate and a right head articulation plate as shown in FIG. 5. These articulation plates are configured to move left and/or head portions of the mattress respectively in response to identify a snoring event by the triggering device.

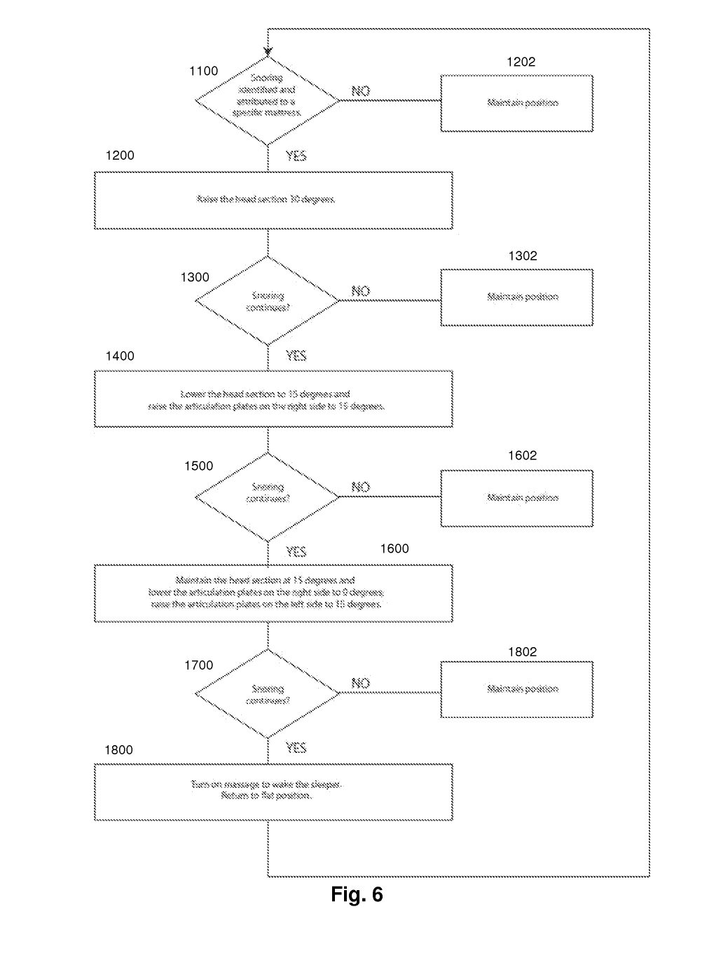

One preferred logic of detecting and reducing a user's snoring of FIG. 4 of the present disclosure is illustrated in FIG. 6. At 1100, the system 10 identifies a snoring event and attributes to a specific mattress by the sensor(s) of the triggering device and the processor. If there is no snoring event detected, at step 1202 the adjustable base portion 26 maintains its position. If there is a snoring event, then at 1200 the adjustable base portion 26 is operated to raise the head articulation plate 38 about 30 degrees. At 1300, the system 10 checks if the snoring continues. If no, at 1302 the adjustable base portion 26 maintains its previous position. If yes, at 1400 the adjustable base portion is operated to lower the head section to about 15 degrees and raise the articulation plates on the right side to about 15 degrees. At 1500, the system 10 checks if snoring continues. If no, at 1602 the adjustable base portion 26 maintains its previous position. If yes, at 1600 the adjustable base portion maintains the head section at about 15 degrees, lowers the articulation plates on the right side to about 0 degrees and raises the articulation plates on the left side to about 15 degrees. At 1700, the system 10 checks if snoring continues. If no, at 1802 the adjustable base portion 26 maintains its previous position. If yes, at 1800, the system 10 may be operated to turn on massage to wake the user and return to flat position.

The method of detecting and reducing a user's snoring preferably further includes detecting another or a subsequent snoring event, further moving one or more articulation plate(s) to move the mattress and thereby changes the sleeping position of the user.

The method may use a software to control the operation steps. The software can be flexible enough to set various options to remedy the problem. The logic/software may change in the sequential order or in degrees (I.e greater or lesser articulation to the left/right/up/down). Artificial intelligence can be incorporated into the software to discern the most effective position in which snoring is alleviated and “learn” to go directly to that position without requiring the user to sequentially go through the process of each step option in the initial software sequence.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for reducing snoring by a user sleeping on a mattress, the system comprising:
a triggering device configured to identify a snoring event; and
an adjustable base portion for supporting the mattress, the adjustable base portion having at least one articulation plate configured to move the mattress to change the sleeping position of the user sleeping on the mattress, in response to the identification of the snoring event by the triggering device.

2. The system according to claim 1 wherein the triggering device comprises a sensing pad on the mattress.

3. The system according to claim 2, wherein the sensing pad is disposed on the mattress.

4. The system according to claim 1, wherein the mattress comprises at least one air filled chamber, and wherein the triggering device comprises at least one sensor in the at least one air filled chamber.

5. The system according to claim 4, wherein the at least one sensor comprises a pressure sensor for measuring changes in the pressure in the at least one air chamber.

6. The system according to claim 1, wherein the triggering device comprises at least one microphone, and a processor for processing signals picked up by the at least one microphone.

7. The system according to claim 6, wherein the at least one microphone comprises a left microphone and a right microphone disposed on the left and right sides of the mattress, and the processor for processing the signals picked up by the left and right microphone configured to determine on which side of the mattress the snoring event is occurring, and moving only the side of the mattress on which the snoring event is occurring.

8. The system according to claim 1, wherein the at least one articulation plate comprises at least one head articulation plate disposed adjacent a head section of the adjustable base portion, a left articulation plate disposed adjacent a left section of the adjustable base portion and a right articulation plate disposed adjacent a right section of the adjustable base portion, for moving the head, left, and right portions, respectively, of the mattress supported on the adjustable base portion.

9. The system according to claim 8 wherein the at least one head articulation plate comprises two head articulation plates, one adjacent the left side of the head section and one adjacent the right side of the head section.

10. The system according to claim 8, wherein the adjustable base portion changes the sleeping position of the user by elevating a portion of the mattress to raise the user's head.

11. The system according to claim 1, wherein the adjustable base portion operates to move at least one of the at least one articulation plates in response to the detection of the snoring event by the triggering device.

12. A method for reducing snoring of a user sleeping on a mattress, supported by a base having at least one articulation plate in contact with the mattress, the method comprising:
identifying a first snoring event;
in response to the identification of a snoring event moving at least one of the at least one articulation plate to move the mattress and thereby change the sleeping position of the user.

13. The method according to claim 12 further comprising: in response to detecting a second or a subsequent snoring event, further moving at least one of the at least one articulation plate to move the mattress and thereby changes the sleeping position of the user.
